(19) United States
(12) Patent Application Publication
(10) Pub. No.: US 2009/0176719 A1
(43) Pub. Date: Jul. 9, 2009

(54) COMPOSITIONS AND METHODS FOR TREATING PERIORAL DERMATITIS

(75) Inventor: Jay Goldstein, Newton, MA (US)

Correspondence Address: Pabst Patent Group LLP, 1545 PEACHTREE STREET NE, SUITE 320, ATLANTA, GA, 30309, US

(73) Assignee: LioLabs LLC, Newton, MA (US)

(21) Appl. No.: 12/349,671

(22) Filed: Jan. 7, 2009

Related U.S. Application Data
(60) Provisional application No. 61/019,469, filed on Jan. 7, 2008.

(51) Int. Cl. A61K 31/7048; A61K 31/65; A61K 31/43; A61K 31/397; A61K 31/545; A61K 31/70; A61K 31/56; A61P 17/00

(52) U.S. Cl. 514/29; 514/154; 514/192; 514/210.15; 514/200; 514/25; 514/171

(57) ABSTRACT

Methods for treating perioral dermatitis are described herein. The method includes administering topically a composition containing an effective amount of a systemic or topical antibiotic and a corticosteroid. The concentration of the antibiotic is from about 0.01% to about 5% by weight of the composition and the concentration of the corticosteroid is from about 0.01% to about 5% by weight of the composition. The composition can contain one or more pharmaceutically acceptable excipients and/or carriers. The compositions can be formulated as a lotion, cream, gel, ointment, paste, powder, solution, suspension, spray, foam, or patch.

### CROSS-REFERENCE TO RELATED APPLICATIONS

This application claims priority to U.S. Ser. No. 61/019,469 entitled “Compositions for Treating Perioral Dermatitis” by Jay Goldstein, filed on Jan. 7, 2008.

### FIELD OF THE INVENTION

This invention is generally in the field of topical formulations for the treatment of perioral dermatitis.

### BACKGROUND OF THE INVENTION

Perioral dermatitis is a skin condition that affects mostly adult women, typically between the ages of 20 and 50. Perioral dermatitis is characterized by papules, and less often pustules, found chiefly on the lower portion of the face, i.e., around the mouth and along the mandibular line. While perioral dermatitis is often classified as a subset of acne, this is not accurate since occurrences of perioral dermatitis lack comedones (i.e., blackheads and whiteheads) which are indicative of acne.

The etiology of perioral dermatitis is unknown. It has been hypothesized that a variety of factors and irritants, such as excess washing of the lower portion of the face; use of fluorinated toothpastes; overuse of topical steroids; use of cosmetics; environmental factors, such as exposure to UV light, heat, and wind; microbiological agents, and/or miscellaneous factors, such hormonal changes, use of oral contraceptives, and gastrointestinal disturbances may cause or exacerbate perioral dermatitis. In the case of fluorinated toothpastes, the papules are usually limited to the perioral region and switching brands of toothpaste or taking care to remove any remaining toothpaste from the perioral region of the face typically results in the disappearance of the papules.

For more serious cases of perioral dermatitis, such as those that affect the mandibular line as well as the perioral region and/or are of unknown etiology, the use of systemic antibiotics had had some success. However, the use of systemic antibiotics is not always successful. Further, the use of systemic antibiotics can require long treatment times in order to control the outbreak and/or can result in adverse side effects.

There exists a need for compositions and methods of use thereof for the treatment of perioral dermatitis which are clinically effective and alleviate the symptoms in a short period of time, with little or no adverse side effects.

Therefore, it is an object of the invention to provide topical compositions for the treat of perioral dermatitis that are clinically effective and alleviate the symptoms of perioral dermatitis in a short period of time.

### SUMMARY OF THE INVENTION

Methods for treating perioral dermatitis are described herein. The method includes administering topically a composition containing an effective amount of a systemic or topical antibiotic and a corticosteroid. Suitable antibiotics include, but are not limited to, tetracyclines, such as doxycycline, demecocycline, minocycline, oxytetracycline, and tetracycline; sulfonamides; quinolones; penicillins; monobactams; macrolides, such as erythromycin, azithromycin, clarithromycin, dirithromycin, roxithromycin, and troleandomycin; cephalosporins; clindamycin; lincoamycin; and metronidazole. Suitable corticosteroids include, but are not limited to, desonide, desoximetasone, mometasone, triamcinolone fluocinolone, hydrocortisone, clocortolone, predincarbate, and aclometasone. The concentration of the antibiotic is from about 0.01% to about 5% by weight of the composition and the concentration of the corticosteroid is from about 0.01% to about 5% by weight of the composition. The composition may also contain one or more pharmaceutically acceptable excipients and/or carriers. The compositions can be formulated as a lotion, cream, gel, ointment, paste, powder, solution, suspension, spray, foam, or patch.

In one embodiment, the composition contains clindamycin hydrochloride at a concentration of 1% by weight of the composition and desonide at a concentration of 0.05% by weight of the composition. In another embodiment, the composition can be administered in varying strengths to treat the initial outbreak and to maintain treatment. For example, a first composition containing 1% clindamycin hydrochloride by weight of the composition and 0.05% desonide by weight of the composition can be administered to treat the initial outbreak, followed by compositions with a decreased concentration of desonide (e.g., 0.025% by weight of the composition and 0.01% by weight of the composition) as the degree of inflammation decreases.

In another embodiment, the composition is in the form of an ointment wherein the active agents are incorporated into an ointment base containing between 1 and up to 30% by weight, more preferably between 1 and 20%, most preferably between about 5 and 10% by weight particles, such as titanium dioxide or a similar material

### DETAILED DESCRIPTION OF THE INVENTION

### I. Definitions

“Perioral dermatitis”, “adult onset acne”, “mandibular acne”, or “perioral dermatitis with mandibular involvement” are used interchangeably and refer to a skin condition characterized by papules, and less often pustules, found chiefly on the lower portion of the face, i.e., around the mouth and along the mandibular line. Perioral dermatitis, as used herein, may include outbreaks resulting from a known etiology, such as an irritant (toothpaste, cosmetics, etc.); however, the outbreaks generally will result from an unknown or undetermined etiology.

### II. Compositions

A. Antibiotics

The compositions can contain a systemic or topical antibiotic. Suitable antibiotics include, but are not limited to, tetracyclines, such as doxycycline, demecocycline, minocycline, oxytetracycline, and tetracycline; sulfonamides; quinolones; penicillins; monobactams; macrolides, such as erythromycin, azithromycin, clarithromycin, dirithromycin, roxithromycin, troleandomycin; cephalosporins; clindamycin; lincoamycin; and metronidazole. The concentration of the antibiotic is from about 0.01% to about 5% by weight of the composition. In one embodiment, the antibiotic is clindamycin hydrochloride having a concentration of about 1% by weight of the composition.

B. Corticosteroids

Suitable corticosteroids include, but are not limited to, desonide, desoximetasone, mometasone, triamcinolone fluocinolone, hydrocortisone, clocortolone, predincarbate, and aclometasone. The concentration of the corticosteroid is from about 0.01% to about 5% by weight of the composition. In one embodiment, the corticosteroid is desonide having a concentration of about 0.05% by weight of the composition. In another embodiment, the corticosteroid is desonide having a concentration of about 0.025% by weight of the composition. In yet another embodiment, the corticosteroid is desonide having a concentration of about 0.01% by weight of the composition.

C. Pharmaceutical Compositions

The antibiotic and the corticosteroid can be combined with one or more pharmaceutically acceptable excipients and/or carriers to form a pharmaceutical composition for topical or transdermal delivery. As would be appreciated by one of skill in this art, the excipients and/or carriers may be chosen based on the dosage form used, the active agents being delivered, the disease or disorder to be treated, etc.

As used herein, the term “pharmaceutically acceptable carrier” means a non-toxic, diluent, encapsulating material or formulation auxiliary of any type. Remington's Pharmaceutical Sciences Ed. by Gennaro, Mack Publishing, Easton, Pa., 1995 discloses various carriers used in formulating pharmaceutical compositions and known techniques for the preparation of such dosage forms.

1. Excipients and Carriers

Suitable excipients include surfactants, emulsifiers, emulsion stabilizers, anti-oxidants, emollients, humectants, chelating agents, suspending agents, thickening agents, occlusive agents, preservatives, stabilizing agents, pH modifying agents, solubilizing agents, solvents, colorants, fragrances, penetration enhancers, and other excipients.

i. Emulsifiers

Suitable emulsifiers include, but are not limited to, straight chain or branched fatty acids, polyoxyethylene sorbitan fatty acid esters, sorbitan fatty acid esters, propylene glycol stearate, glyceryl stearate, polyethylene glycol, fatty alcohols, polymeric ethylene oxide-propylene oxide block copolymers, and combinations thereof.

ii. Surfactants

Suitable surfactants include, but are not limited to, anionic surfactants, non-ionic surfactants, cationic surfactants, and amphoteric surfactants. Examples of anionic surfactants include, but are not limited to, ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinate, disodium lauryl sulfosuccinate, diammonium lauryl sulfosuccinate, tetrasodium N-(1,2-dicarboxyethyl)-N-octadecylsulfosuccinate, diamyl ester of sodium sulfosuccinic acid, dihexyl ester of sodium sulfosuccinic acid, dioctyl esters of sodium sulfosuccinic acid, docusate sodium, and combinations thereof.

Examples of nonionic surfactants include, but are not limited to, polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethylene oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and combinations thereof.

Examples of amphoteric surfactants include, but are not limited to, sodium N-dodecyl-ÿ-alanine, sodium N-lauryl-ÿ-iminodipropionate, myristoamphoacetate, lauryl betaine, lauryl sulfobetaine, sodium 3-dodecyl-aminopropionate, sodium 3-dodecylaminopropane sulfonate, sodium lauroamphoacetate, cocodimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine, and combinations thereof.

Examples of cationic surfactants include, but are not limited to, behenyl trimethyl ammonium chloride, bis(acyloxyethyl)hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowedimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picolin benzyl ammonium chloride, polyquatemium, stearalkonium chloride, sterayl dimethylbenzyl ammonium chloride, stearyl trimethyl ammonium chloride, trimethylglycine, and combinations thereof.

iii. Suspending Agents

Suitable suspending agents include, but are not limited to, alginic acid, bentonite, carbomer, carboxymethylcellulose and salts thereof, colloidal oatmeal, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and combinations thereof.

iv. Antioxidants

Suitable antioxidants include, but are not limited to, butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and combinations thereof.

v. Chelating Agents

Suitable chelating agents include, but are not limited to, EDTA, disodium edetate, trans-1,2-diaminocyclohexane-N,N,N′,N′-tetraaceticacid monohydrate, N,N-bis(2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′,N′-tetrakis(methylenephosphonic acid), O,O′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, nitrilotripropionic acid, nitrilotris(methylenephosphonic acid), 7,19,30-trioxa-1,4,10,13,16,22,27,33-octaazabicyclo[111,11,1]pentatriacontane hexahydrobromide, triethylenetetramine-N,N,N′,N″,N′″,N′″-hexaacetic acid, and combinations thereof.

vi. Emollients

Suitable emollients include, but are not limited to, myristyl lactate, isopropyl palmitate, light liquid paraffin, cetearyl alcohol, lanolin, lanolin derivatives, mineral oil, petrolatum, cetyl esters wax, cholesterol, glycerol, glycerol monostearate, isopropyl myristate, lecithin, and combinations thereof.

vii. Humectants

Suitable humectants include, but are not limited to, glycerin, butylene glycol, propylene glycol, sorbitol, triacetin, and combinations thereof.

viii. pH Modifying Agents

The compositions described herein may further contain sufficient amounts of at least one pH modifier to ensure that the composition has a final pH of about 3 to about 11. Suitable pH modifying agents include, but are not limited to, sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and combinations thereof.

ix. Preservatives

Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, thimerosal, and combinations thereof.

D. Dosage Forms

The compositions described herein can be formulated for topical or transdermal administration. Suitable dosage forms for topical or transdermal administration include ointments, pastes, creams, lotions, gels, ointments, pastes, powders, solutions, sprays, foams, or transdermal device, such as patches. Methods for making these dosage forms are well known in the art. See, for example, Ansel, Popovich, and Allen, Pharmaceutical Dosage Forms and Drug Delivery Systems 6^{th} Ed., Williams and Wilkins, 1995.

1. Creams, Pastes, and Ointments

i. Creams

Creams are generally characterized as semisolid dosage forms formed from an oil-in-water emulsion, a water-in-oil emulsion, or an aqueous microcrystalline dispersion. Creams are generally less viscous and lighter than ointments. Creams are considered to have greater esthetic appeal than ointments or pastes due to their non-greasy character and their ability to “vanish” into the skin upon rubbing.

ii. Ointments

Ointments are prepared by mixing one or more active agents in an ointment base. The ointment base is semisolid and can be either hydrophobic or hydrophilic. Suitable ointment bases include hydrocarbon bases, such as petrolatum, white petrolatum, yellow ointment, and mineral oil; absorption bases, such as hydrophilic petrolatum, anhydrous lanolin, lanolin, and cold cream; water-removable bases, such as hydrophilic ointments; and water-soluble bases, such as polyethylene glycol ointments. Selection of the ointment base is dependent on a number of factors, such as the desired release rate of the active agent(s) from the ointment base; the desirability for enhancement by the base of the percutaneous absorption of the active agent(s); the advisability of occlusion of moisture from the skin by the base; the short-term and long-term stability of the active agent(s) in the base; and the influence, if any, of the active agent(s) on the consistency or other features of the ointment base.

In one embodiment, the composition is in the form of an ointment wherein the active agents are incorporated into a pharmaceutically acceptable ointment base containing between 1 and up to 30% by weight, more preferably between 1 and 20%, most preferably between about 5 and 10% by weight particles.

Suitable bases include, but are not limited to, petrolatum, mineral oil, vegetable oils, and combinations thereof. In a preferred embodiment, the particles are titanium dioxide and the base is petrolatum.

Suitable particles include zinc oxide, such as an ultrafine grade of micronized zinc oxide, magnesium oxide, talc, and combinations thereof. In a preferred embodiment, the particles are titanium dioxide. The titanium dioxide used to prepare the formulations generally has a diameter less than about 100 microns, preferably between 10 nm and 100 microns. In one embodiment, the diameter of the titanium dioxide particles is 44 microns. In another embodiment, the diameter of the titanium dioxide particles is 0.3 microns. In still another embodiment, the diameter of the titanium dioxide particles is 15 nm (0.015 microns).

iii. Pastes

Pastes contain more solid materials than ointments and are therefore stiffer and less penetrating. Pastes are usually employed for their protective action and for their ability to absorb substantial discharges from skin lesions or pustules due to their ability to stay in place after application with little tendency to soften and flow. Because of their large percentage of solids, pastes are generally more absorptive and less greasy than ointments prepared from the same components.

2. Lotions

While creams, pastes, and ointments are classified as semisolid preparations, lotions are characterized as liquids. Lotions are generally suspensions of solid materials (such as active agents) in an aqueous vehicle, although certain emulsions and even some true solutions have been designated as lotions due to their appearance or application. Lotions may be preferred over semisolid formulations due to their non-greasy character and their increased spreadability over large areas of skin. Lotions typically contain finely powdered substances that are insoluble in the dispersion medium and are suspended through the use of suspending agents and dispersing agents. Other lotions can have as the dispersed phase liquid substances that are immiscible with the vehicle and are usually dispersed by means of emulsifying agents or other suitable stabilizers,

3. Powders

Medicinal powders are mixtures of one or more active agents with a solid inert base, such as talcum powder. Depending upon the particle size of the resulting blend, the powder will have varying dusting and covering capabilities. The particle size should be small enough to ensure against grittiness and consequent skin irritation.

4. Foams

Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Foams may be formulated in various ways to provide emollient or drying functions to the skin, depending on the formulation constituents.

Foams may contain an emulsion. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

The oil phase can be prepared by mixing together the surfactant(s) and emulsifier(s) and melting. The aqueous phase is prepared separately by dissolving the preservatives in water with heating. The aqueous phase is added to the oil phase with continuous high shear mixing to produce a milky emulsion. The emulsion is cooled and the pH is adjusted by the addition of a buffer. The active agent can be either pre-dissolved in aqueous or organic phase or suspended/dispersed in the final emulsion.

Foams generally contain a pharmaceutically acceptable propellant. Suitable propellants include, but are not limited to, hydrofluoroalkanes, hydrofluorocarbons, volatile alcohols, hydrocarbon gases, and combinations thereof. Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable.

The emulsion concentrate is placed in pressure cans, preferably coated aluminum cans to prevent corrosion, such as epoxy-coated cans. The lid and dispensing apparatus are crimped in place. The can is charged with propellant to the stated level, for example, by adding 30 grams of propellant per 70 grams of emulsion. At the time of application, the mixture of the emulsion with the propellant may be insured by shaking, optionally with the aid of a mixing bead. The dispenser may be metered or unmetered (continuous). Metered dispensing is preferred for highly active materials such as hydrocortisone and other steroids. The can may be arranged for either “upside down” spraying with the valve at the bottom, or the can have a dip tube so that the foam can be sprayed while the can is upright with the valve at the top.

### III. Methods of Use

The topical formulations herein can be used to treat perioral dermatitis or known or unknown etiology. The amount of the composition to be administered and the frequency of administration can readily be determined by the treating physician based on a variety of factors, such as the condition to be treated, the severity of the condition, and the age and weight of the patient to be treated. Perioral dermatitis occurs primarily in adult women, typically between the ages of 20 and 50. It is unusual for the disorder to appear in men or teenage girls (unlike acne). While sever case may respond to systemic or topical antibiotics, the addition of a topical corticosteroid, such as desonide, to a topical antibiotic greatly shortens the time necessary to achieve control and increases the efficacy of the treatment. The use of a corticosteroid is counter intuitive since one of the common effects of steroids is the development of acne and acneform eruptions (known as steroid acne).

Compositions of varying strength can be administered depending on the degree of inflammation. For example, a composition containing 1% clindamycin and 0.05% desonide would be administered initially. Once control of the outbreak has begun, a composition containing 1% clindamycin and 0.025% desonide would be administered. Finally, a composition containing 1% clindamycin and 0.01% desonide would be administered as a maintenance dosage.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for the treatment of perioral dermatitis comprising administering topically a composition comprising an effective amount of at least one antibiotic and at least one corticosteroid.

2. The method of claim 1, wherein the antibiotic is a systemic or topical antibiotic.

3. The method of claim 2, wherein the antibiotic is selected from the group consisting of tetracyclines, such as doxycycline, demecocycline, minocycline, oxytetracycline, and tetracycline; sulfonamides; quinolones; penicillins; monobactams; macrolides, such as erythromycin, azithromycin, clarithromycin, dirithromycin, roxithromycin, and troleandomycin; cephalosporins; clindamycin; lincoamycin; and metronidazole.

4. The method of claim 1, wherein the corticosteroid is selected from the group consisting of desonide, desoximetasone, mometasone, triamcinolone fluocinolone, hydrocortisone, clocortolone, predincarbate, and aclometasone.

5. The method of claim 1, wherein the antibiotic is clindamycin and the corticosteroid is desonide.

6. The method of claim 5, wherein the concentration of clindamycin is from about 0.01% to about 5% by weight of the composition and the concentration of desonide is from about 0.01% to about 5% by weight of the composition.

7. The method of claim 6, wherein the concentration of clindamycin is about 1% and the concentration of desonide is about 0.05%.

8. The method of claim 6, wherein the concentration of clindamycin is about 1% and the concentration of desonide is about 0.025%.

9. The method of claim 6, wherein the concentration of clindamycin is about 1% and the concentration of desonide is about 0.01%.

10. The method of claim 1, wherein the composition further comprises one or more pharmaceutically acceptable excipients.

11. The method of claim 1, wherein the composition is formulated as a lotion, cream, gel, ointment, paste, powder, solution, suspension, spray, foam, or patch.
